(19) United States
(12) Patent Application Publication
(10) Pub. No.: US 2011/0171230 A1
(43) Pub. Date: Jul. 14, 2011

(54) Method of Identifying Agents that Promote Axonal Development

(75) Inventors: Paul Frederic Worley, Baltimore, MD (US); Guo-li Ming, Clarksville, MD (US); Joseph P. Steiner, Mount Airy, MD (US)

(73) Assignee: The Johns Hopkins University, Baltimore, MD (US)

(21) Appl. No.: 12/989,498

(22) PCT Filed: Apr. 24, 2009
(86) PCT No.: PCT/US09/41717

§ 371 Date: Mar. 14, 2011

Related U.S. Application Data
(60) Provisional application No. 61/125,528, filed on Apr. 25, 2008.

(51) Int. Cl. A61K 39/395; G01N 33/573; C12Q 1/68; C12N 9/99; A61K 38/02; A61K 31/7088; A61K 31/713; A61P 25/00; A61P 25/28; A61P 25/16; C07K 2/00; C07K 16/40; C07H 21/00; C07H 21/02

(52) U.S. Cl. 424/158.1; 435/7.1; 435/6.1; 435/7.21; 435/184; 514/17.7; 514/44 A; 514/44 R; 530/350; 530/300; 530/389.1; 536/23.1; 536/24.5

(57) ABSTRACT

The present invention is based on the discovery of a novel function of an FKBP proline isomerases in chemotropic nerve guidance and axon regeneration through gating of TRPC1 channel activity. Accordingly, there are provided methods for identifying an agent that inhibits FKBP52 binding to TRPC1. Also provided are methods for identifying an inhibitor of an FKBP52-TRPC1 signaling pathway by contacting a cell expressing TRPC1 and FKBP52 with a test agent and detecting a change in agonist-induced calcium flux of TRPC1. Further provided are methods of enhancing axonal outgrowth by inhibiting the activity of FKBP52 in a neuronal growth cone, and methods of enhancing axonal regeneration in a subject in need thereof by administering an agent that inhibits the activity of FKBP52.

### FIELD OF THE INVENTION

The present invention relates generally to molecules involved in axonal regeneration, and more specifically to molecules that inhibit FKBP52, and methods of identifying or using such molecule to enhance axonal regeneration.

### BACKGROUND OF THE INVENTION

Immunophilins, including families of cyclosporin-binding cyclophilins, parvulins, and FK506-binding proteins (FKBPs), are protein chaperones with peptidyl-prolyl (proline) isomerase activity. These isomerases catalyse the isomerization of peptide proline residues between cis and trans conformations, which can be a rate limiting process that influences protein folding and function. FKBPs and cyclophilins are collectively referred to as immunophilins since they were originally discovered as the biological receptors for the commonly used immunosuppressant drugs FK506 and cyclosporine A, respectively. Though both cyclosporine A and FK506 inhibit isomerase activity of immunophilins, their immunosuppressive function is isomerase activity-independent, and instead results from inhibition of calcineurin by the cyclophilins-cyclosporine A or FKBP-FK506 complex.

Interestingly, FKBPs are 10-50 times more enriched in the nervous system than in the immune system. Previous studies of FKBPs in the nervous system have been largely focused on their roles as calcineurin inhibitors and protein chaperones, functions that are independent of the isomerase activity. While important in clinical applications, these drug-dependent properties of FKBPs are not indicative of the physiological relevance since mammalian cells do not naturally encounter immunosuppressant drugs. The endogenous cellular functions of proline isomerases and their substrate(s) in the nervous system remain to be identified.

### SUMMARY OF THE INVENTION

The present invention is based on the discovery of a novel function of an FKBP proline isomerases in chemotropic nerve guidance and axon regeneration through gating of TRPC1 channel activity. Accordingly, in one embodiment of the invention, there are provided methods for identifying an agent that inhibits FKBP52 binding to TRPC1 by comparing binding of FKBP52 to TRPC1 in the presence and absence of a test agent, wherein a decrease in binding in the presence of test compound is indicative of an agent that is an inhibitor of FKBP52 binding. In some embodiments, the FKBP52 and TRPC1 are expressed in the same cell. In one aspect, binding is determined by co-immunoprecipitation of FKBP52 and TRPC1. In certain embodiments, the test agent may be a peptide, a protein, an antibody, a polynucleotide, an antisense RNA molecule, an RNAi molecule, an siRNA molecule, a peptidomimetic, a chemical compound, a small organic molecule, or an agent from a library of test agents.

In another embodiment of the invention, there are provided methods for detecting or identifying an inhibitor of an FKBP52-TRPC1 signaling pathway by contacting a cell expressing TRPC1 and FKBP52 with a test agent and detecting a change in agonist-induced calcium flux of TRPC1. A decrease in calcium flux in the presence of the test agent as compared to in the absence of test agent, is indicative that the agent is an inhibitor of FKBP52-TRPC signaling. In some embodiments, TRPC1 is activated by a G-protein coupled receptor (GPCR). In particular embodiments, the GPCR is activated by an agonist. In one aspect, the GPCR is a purinergic receptor. In a further aspect, the GPCR is a purinergic receptor and the agonist is UTP. In certain embodiments, the cell is an HEK293 cell.

In a further embodiment of the invention, there are inhibitors of FKBP52 activity, such as isomerase activity, binding of FKBP52 to TRPC1, and/or FKBP52-TRPC1 signaling. Such inhibitors may be identified by the methods described herein.

In another embodiment of the present invention, there are provided, methods of enhancing axonal outgrowth by inhibiting the activity of FKBP52 in a neuronal growth cone, thereby enhancing axonal outgrowth.

In still another embodiment of the present invention, there are provided methods of enhancing axonal outgrowth in a subject in need thereof by administering an agent that inhibits the activity of FKBP52, thereby enhancing axonal outgrowth. In certain embodiments, the FKBP52 activity is isomerase activity or binding of FKBP52 to TRPC1. In one aspect, FKBP52-TRPC1 signaling is inhibited. In certain embodiments, the subject has suffered an injury to the CNS. In other embodiments, the subject has suffered an injury to a peripheral nerve. In some embodiments, the injury is an iatrogenic injury to a nerve during surgery. In one aspect, the surgery is prostate surgery. In other embodiments, the subject has a neurological disorder. In some embodiments, the neurological disorder is a demyelinating disease. In one aspect, the demyelinating disease is multiple sclerosis. In particular embodiments, the neurological disorder is selected from the group consisting of Alzheimer's, Parkinson's disease, senile dementia, memory disturbances/memory loss, Huntington's disease, Lou Gehrig's disease, multiple sclerosis, cerebral palsy, Creutzfeldt-Jakob disease, Niemann Pick disease, and Pick's disease.

### BRIEF DESCRIPTION OF THE DRAWINGS

FIG. 1A shows a schematic diagram of human TRPC1 indicating regions critical for FKBP52 and Homer binding. C-terminal Homer binding site is in in TRP box 2 (PPPF). FIG. 1B shows results of immunoprecipitation of lysates of HEK293 cells expressing HA-TRPC1 constructs (C1) assayed for co-IP with native FKBP52 or Homer 3 (H3). FIG. 1C shows results of immunoprecipitation of lysates of HEK293 cells expressing HA-TRPC1 NT constructs assayed for binding to GST-FKBP52.

FIG. 2A shows immunoblots of lysates of HEK293 cells expressing HA-TRPC1 (C1) alone (10 μg), or with HA-FKBP12 (cDNA at 1 μg, 3, μg, 10 μg) or HA-FKBP52 (cDNA at 0.1 μg, 0.3 μg, 1 μg) assayed for co-IP with native Homer 3 (H3). FIG. 2B shows immunoblots of HEK293 cell lysates assayed for binding in vitro as in (a). FIG. 2C shows immunoblots of HEK293 cells expressing TRPC1 treated with FK506 (150 nM) for indicated times, and lysates assayed for binding, suggesting FK506 increases TRPC1 binding to GST-Homer 3. FIG. 2D shows immunoblots of HEK293 cells transfected with HA-TRPC1 (C1) and FKBP52 and treated with GPI-1046 (1 nM, 100 nM, 500 nM, and 1 μM) for 30 min; lysates were assayed for binding to GST-H3 EVH, indicating GPI-1046 increases TRPC1 binding to GST-Homer. FIG. 2E shows an immunoblot of FKBP52 (FD67DV) co-expressed with TRPC1 and assayed for co-IP with native H3, indicating that isomerase activity of FKBPs is not required to displace Homer. Note that FKBP52 (FD67DV) also co-IPs with Homer, while FKBP52-WT does not.

FIGS. 3A-F show sample current traces for TRPC1 channel opening in HEK293 cells co-transfected with GFP, TRPC1 (A), TRPC1 and FKBP12-WT (B) or FKBP12-D37L (C), TRPC1 and FKBP52-WT (D) or FKBP52-FD67DV (E). FIG. 3F shows a plot of the summary of the spontaneous and receptor-activated current.

FIGS. 4A-C show bright-field and fluorescence images of growth cones of Xenopus spinal neurons loaded with Fluo-4 at different time points after stimulation with netrin-1 (5 ng/ml). Representative images show neurons from an un-injected embryo without (control, A) or with GPI-1046 (500 nM, B) in the bath, or from an FKBP52-FD67DV expressing neuron (C). FIG. 4D shows a plot of the time course of netrin-1-induced Ca²⁺ rise in growth cones under different conditions. FIG. 4E shows a plot of the summary of netrin-1-induced Ca²⁺ changes in growth cones under different conditions.

FIGS. 5A-D show images of the growth cone turning responses in a gradient of netrin-1 by neurons in the control medium (A), in the presence of bath application of FK506 (100 nM, B) or GPI-1046 (100 nM, C), and neurons derived from embryos injected with the mRNA encoding FKBP52-FD67DV (D), respectively. The left two columns of images show growth cones at the start (0′) and the end of exposure (30′) in a netrin-1 gradient. The right column shows superimposed trajectories of neurite extension during the 30′ period for a sample population of 12 neurons under each condition. The origin is the center of the growth cone and the original direction of growth is vertical. Arrows indicate the direction of the gradient. FIGS. 5E-F show plots of the summary of turning angles under different conditions.

FIG. 6A shows a schematic diagram of commissural interneuron projections in the developing Xenopus spinal cord. FIGS. 6B-I show representative images of sagittal view of commissural interneurons and their axonal projections in the Xenopus spinal cord from stage 25 embryos. Shown are projections of Z-stack confocal images of 3A10 immunostaining of commissural interneuron axons from un-injected embryos (control, B), with treatment of GPI-1046 (0.5 μM, D) or FK506 (0.5 μM, E), or embryos injected with XDCC-MO (C), FKBP52-WT (F), FKBP52-FD67DV (G), TRPC1-WT (H) or TRPC1-P645L (I). FIG. 6J shows a plot depicting the quantification of the percentage of 3A10⁺ commissural interneurons with normal midline crossing under different experimental conditions.

FIG. 7A shows images of growth cone responses in a gradient of MAG of primary hippocampal neurons expressing GFP alone, or co-expressing GFP and FKBP52-FD67DV, TRPC1-P645A, TRPC1-P645A and FKBP52-WT. FIG. 7B shows a plot of the summary of growth cone turning angles in response to a MAG gradient under different conditions.

### DETAILED DESCRIPTION OF THE INVENTION

The present invention is based on the discovery that FKBP52 exhibits competitive binding to TRPC1 with homer, and ligand-induced channel opening of TRPC1 depends on isomerase activity of FKBP52. Functionally, inhibiting isomerase activity of FKBP52 or expressing a TRPC1 mutant defective in interaction with FKBP52 abolishes netrin-1-induced Ca²⁺ rise and subsequent turning of neuronal growth cones in vitro and leads to defects in netrin-1-dependent midline axon guidance of commissural interneurons in the developing spinal cord in vivo. Furthermore, isomerase activity of FKBP52 is required for growth cone responses to myelin-associated glycoprotein, an inhibitor for axon regeneration. The studies provided herein demonstrate a novel function of proline isomerases in chemotropic nerve guidance and axon regeneration through gating of TRPC1 channel activity. These findings also have significant implications for clinical applications of immunophilin-related therapeutic drugs.

In one embodiment of the invention, there are provided methods for identifying an agent that inhibits FKBP52 binding to TRPC1 by comparing binding of FKBP52 to TRPC1 in the presence and absence of a test agent, wherein a decrease in binding in the presence of test compound is indicative of an agent that is an inhibitor of FKBP52 binding. In one aspect, binding is determined by co-immunoprecipitation of FKBP52 and TRPC1. In certain embodiments, the test agent may be a peptide, a protein, an antibody, a polynucleotide, an antisense RNA molecule, an RNAi molecule, an siRNA molecule, a peptidomimetic, a chemical compound, a small organic molecule, or an agent from a library of test agents.

FKBP52 is a mammalian FKBP with multiple domains: two FKBP domains with detectable rotamase or peptidylprolyl cis-trans isomerase (PPIase) activity, a third domain, which contains three tetratricopeptide repeat motifs, followed by a calmodulin-binding consensus sequence. The N-terminal domain of FKBP52 (FKBP52-N; residues 1-140) is responsible for PPIase activity and binding of FK506. The crystal structure of FKBP52-N has been determined by molecular replacement to 2.4 A. Additional structural determinations of regions of overlap in FKBP52 have also been crystallized. FKBP52-N is defined by a six-stranded anti-parallel beta-sheet wrapping with a right-handed twist around a short alpha-helix, a structure similar to that of FKBP12. Thus, FKBP52-N is able to bind FK506 in a similar way to FKBP12. The principal reason for the specificity differences between FKBP52-N and FKBP12 is the variability in two loop regions (residues 70-76 and 108-127). The Pro 120 change corresponding to Gly89 in FKBP12 limits the conformational adaptation between the loop (residues 108-127) and FK506 and decreases the FK506 affinity, while the Lys121 substitution corresponding to Ile90 of FKBP12 destroys a key interaction between FKBP52-N and calcineurin. As described above, the locations of strictly conserved amino acids in the polypeptide chain provide for the overall conformation of the PPIase domains of FKBPs is essential for the PPIase activity.

The family of canonical TRP channels (TRPC) are six membrane spanning cation channels that mediate diverse cellular responses in neurons, including sensory transduction, G-protein receptor signaling, and axonal outgrowth and guidance.

In some embodiments, the FKBP52 and TRPC1 are expressed in the same cell. In certain embodiments, the cell is transfected with a nucleic acid molecule encoding FKBP52, a nucleic acid molecule encoding TRPC1, or both. In one aspect, the cell is transfected with a nucleic acid encoding both FKBP52 and TRPC1. Cells may be transiently or stably transfected.

In another embodiment of the invention, there are provided methods for detecting or identifying an inhibitor of an FKBP52-TRPC1 signaling pathway by contacting a cell expressing TRPC1 and FKBP52 with a test agent and detecting a change in agonist-induced calcium flux of TRPC1. A decrease in calcium flux in the presence of the test agent as compared to in the absence of test agent, is indicative that the agent is an inhibitor of FKBP52-TRPC signaling. TRPC1 channels can be activiated by receptors of various types, for example, G-protein coupled receptors (GPCRs). Thus, activation of such GPCRs by application of an agonist thereof can activate a TRPC1 channel. Thus, as used herein “agonist-induced current” or “agonist-induced flux” refers to current or flux due to the activation of TRPC1 via activation of a GPCR via application of an agonist for that GPCR. As would be recognized by a skilled artisan, any receptor shown to activate a TRPC1 channel may be used in the invention methods. In one aspect, the GPCR is a purinergic receptor. In one example, the GPCR is a purinergic receptor and the agonist is UTP. In another aspect, the GPCR is a metabotropic glutamate receptor (mGluR), such as a Group I mGluR. In one example, the mGluR is mGluR1.

In certain embodiments, the change in agonist-induce calcium influx may be detected as a change in current using electrophysiological methods known in the art. For example, currents may be recorded in a whole-cell current recording configuration using cells expressing FKBP52 and TRPC1 proteins (Huang et al., Nat Cell Biol 8:1003-10, 2006; and Yaun et al., Nat Cell Biol 9:636-45, 2007).

Ca²⁺ signalling has been implicated in signalling of myelin and glial scar associated inhibitors that prevent axonal regeneration in the adult central nervous system. It has been shown that TRPC-dependent Ca²⁺ influx is required for repulsive and inhibitory growth cone responses to myelin-associated inhibitors, including Myelin-associated glycoprotein (MAG), which is a major component of myelin. Studies showed that axonal growth cones of hippocampal neurons exhibited significant repulsive turning responses in a MAG gradient. Application of the non-immunosuppressant FKBP inhibitor, GPI-1046 or over-expression of a mutant of FKBP that lacks isomerase activity (FKBP52-FD67DV) abolished MAG-induced repulsion in these neurons. Furthermore, over-expression of TRPC1-P645A, which shows constitutive channel activity, also blocked MAG-induced growth cone repulsion. TRPC1-P645A is additionally notable in that its channel opening can be rescued by over expression of FKBP52. Co-expression of FKBP52-WT with TRPC1-P645A rescued MAG-induced growth cone repulsion in neurons. These results suggest that proline isomerasation of TRPC1 by FKBP52 essential for transducing MAG signalling.

Regulated activity of TRPC1 is required for repulsive response of growth axons to myelin basic protein (MAG). MAG is a major inhibitor of axonal outgrowth, and inhibition of MAG evoked TRPC1 prevents the repulsive response to MAG. As provided herein, FKBP12 and the related FKBP52 enhance TRPC1 channel opening. Importantly, FKBP52 is specifically required for receptor activated TRPC1 activity while another FKBP protein, FKBP12, mediates stimulus-independent channel opening. The relative actions of FKBP12 and FKBP52 define the magnitude of basal and stimulus-dependent responses, respectively. Because FKBP12 and FKBP52 are both highly expressed in neurons, FKBP inhibitors that are optimized for FKBP12 can shift the balance toward FKBP52, which preserves or even enhances TRPC1 channel responsiveness to MAG. This suggests that FKBP52 selective agents that are optimized for there ability to block FKBP52 binding to TRPC1 will effectively enhance axonal regeneration. Structural data indicate that the binding pockets of FKBP12 and FKBP52 are sufficiently distinct as to support the development of selective agents.

As provided herein, studies using biochemistry, electrophysiology, Ca²⁺ imaging and functional axon guidance assays in vitro and in vivo, a novel physiological function of proline isomerases in the nervous system has been demonstrated. While not wishing to be bound by any particular theory, these data support a model that isomerase function of FKBP52 controls axon guidance and regeneration through its modulation of TRPC channel opening and subsequent Ca²⁺ rise.

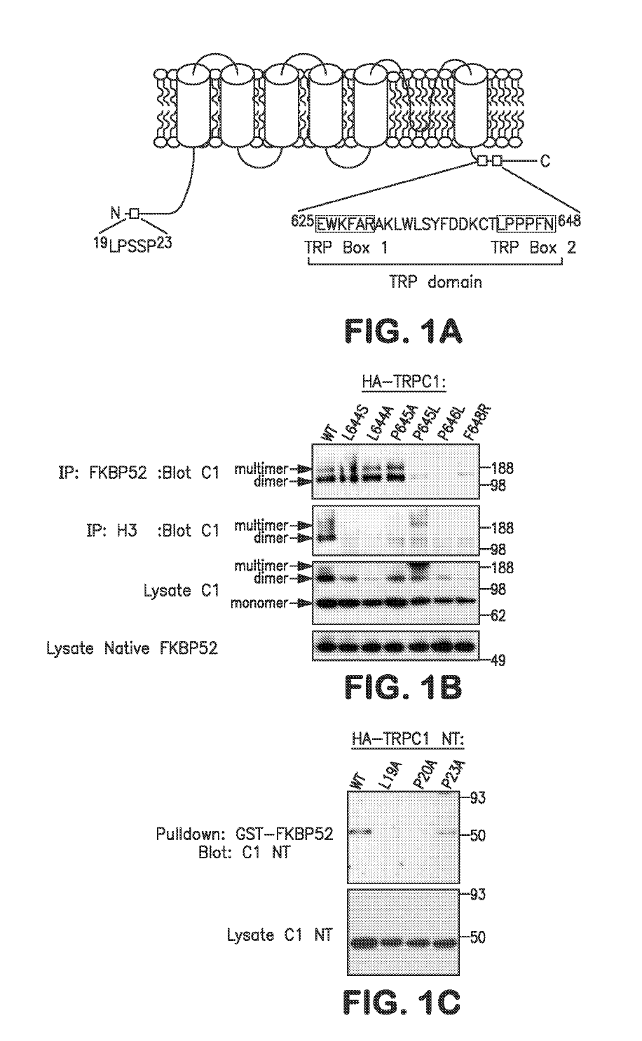

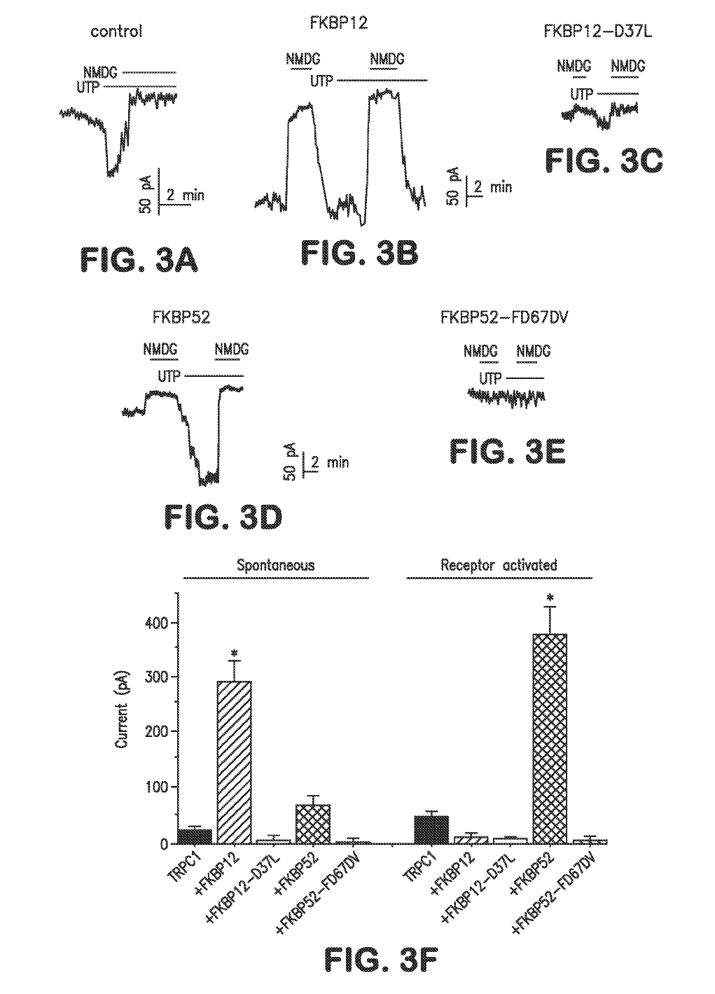

In particular, it is shown herein that proline isomerase activity of both FKBP52 and FKBP12 regulate the channel activity of TRPC1, but these proteins display diametrically distinct actions. Specifically, FKBP12 causes the channel to be constitutively open, while FKBP52 enhances stimulus-dependent channel opening (FIG. 3). It has been demonstrated that Homer-dependent cross-linking of the N- and C-termini of TRPC1 prevents spontaneous channel activity, and that the immediate early gene form of Homer (H1a), which acts as a dominant negative to interrupt Homer cross-linking, confers dynamic control of spontaneous TRPC activity (Yuan et al., Cell 114:777-89, 2003). Like Homer, FKBP52 can bind TRPC1 at both the N- and C-terminus and dimerize (FIG. 1), and these properties may be critical for its distinct action. The data provided herein suggest that the TRPC1-FKBP52 complex undergoes rapid isomerization in response to changing physical interactions with other proteins, such as Stim1 or IP3R, as a consequence of agonist stimulation or store depletion. Because FKBP12 increases spontaneous activity while FKBP52 enhances agonist-dependent channel opening, the coordinated action of Homer and FKBPs on TRPC1 channels may provide a fine-tuning mechanism for Ca²⁺ dynamics, which may underlie the changes of growth cone motility. Notably, this model is the first example of a requirement for proline isomerase activity in a rapid signalling event. Provided herein is evidence to support the role of FKBP52-TRPC association in commissural axon guidance in vivo.

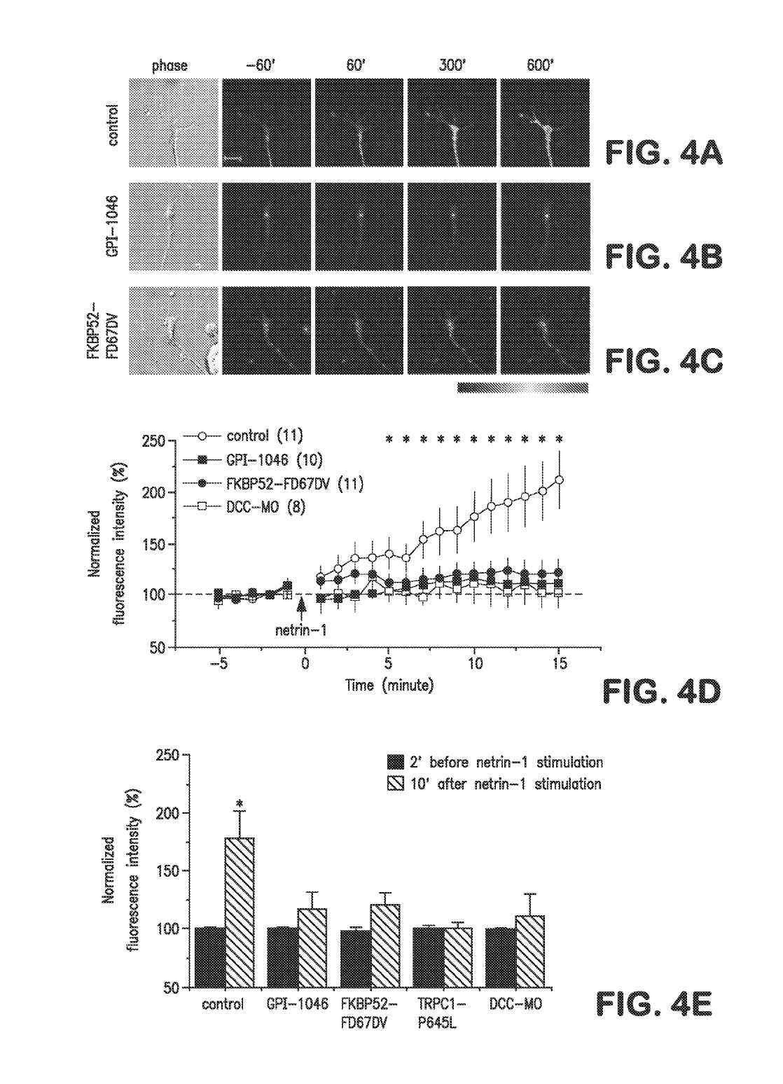

Both basal level and ligand-induced elevation of Ca²⁺ are essential for setting the polarity of growth cone steering in response to guidance cues (Hong et al., Nature 403, 93-8, 2000; Zheng et al., Nature 403, 89-93, 2000; Gomez & Zheng, Nat Rev Neurosci 7, 115-25, 2006; and Zheng & Poo, Annu Rev Cell Dev Biol 23, 375-404, 2007). The blockade of FKBP52-dependent agonist-stimulated current (FIG. 3F) and netrin-1-induced Ca²⁺ by isomerase inhibition of FKBP52 (FIG. 4) suggests that isomerase regulation of TRPC1 is ligand-dependent. It was also found that in Xenopus spinal neurons FKBP52 promotes netirn-1-induced Ca²⁺ influx through XTRPC1. Interestingly, the FKBP52 binding sites in TRPC1 are conserved among members of the TRPC family, but not in TRPV families, thus the binding region or the target proline residue in TRPs may differ and the channel activity may be differentially regulated by FKBPs.

The terms “test agent” or “candidate agent” are used interchangeably herein and are used broadly herein to mean any agent that is being examined for inhibitor activity in a method of the invention. Although the methods generally are used as screening assays to identify previously unknown molecules that can act as inhibitors as described herein, the methods also can be used to confirm that a agent known to have a particular activity in fact has the activity. Candidate compounds that affect the binding of FKBP52 to TRPC1, the isomerase activity of FKBP52, or the FKBP52-TRPC1 signaling pathway include peptides, proteins, antibodies, polynucleotides, antisense RNA, RNAi, siRNA, peptidomimetics, chemical compounds, small organic molecules, or can be one of a plurality of similar but different agents (e.g., a library of test agents such as a combinatorial library of test agents, which can be a randomized or biased library or can be a variegated library based on known effective agent).

In some embodiments, the test agent is a chemical compound. One class is organic molecules, for example small organic compounds having a molecular weight of more than 50 and less than about 2,500 Daltons. Test agents may include functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups.

The test agent may also be a combinatorial library for screening a plurality of compounds. Compounds such as peptides identified in the method of the invention can be further cloned, sequenced, and the like, either in solution of after binding to a solid support, by any method usually applied to the isolation of a specific DNA sequence Molecular techniques for DNA analysis (Landegren et al., Science 242:229-237, 1988) and cloning have been reviewed (Sambrook et al. Molecular Cloning: a Laboratory Manual, 2nd ed.; Cold Spring Harbor Laboratory Press, Plainview, N.Y., 1998, herein incorporated by reference).

Candidate compounds may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

A variety of other agents may be included in the screening assay. These include agents like salts, neutral proteins, e.g., albumin, detergents, etc. that are used to facilitate optimal protein-protein binding and/or reduce nonspecific or background interactions. Reagents that improve the efficiency of the assay, such as protease inhibitors, nuclease inhibitors, antimicrobial agents, etc., may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 10 hours will be sufficient.

“Incubating” includes conditions which allow contact between the test agent and the a protein or proteins or a cell expressing a protein or proteins of interest (e.g., FKBP52 and TRPC1). “Contacting” includes in solution or in solid phase and may be in vitro or in vivo.

Suitable cells for use in the invention methods include any host cells expressing an FKBP52 protein and a TRPC1 protein. The cells can be primary cells or cells of a cell line. In one embodiment, the sample is a primary cell, such as a neuronal cell, that expresses an FKBP52 protein and a TRPC1 protein. In another embodiment, the cell is a cell line that expresses an FKBP52 protein and a TRPC1 protein. Specific, non-limiting examples of cells suitable for use with the method of the invention are cultured hippocampal neurons or spinal neurons. Methods of culturing a neuronal cell suitable for use in the method of the invention are known to one of skill in the art (see O'Brien et al., Neuron 21:1067-98, 1998; O'Brien et al., Curr. Opin. Neurobiol. 8:364-9, 1998; O'Brien et al., J. Neurosci. 17:7339-50, 1997; Mammen et al., J. Neurosci. 17: 7351-8, 1997; Liao et al., Nature Neurosci. 2:37-43, 1999, all herein incorporated by reference).

In yet another embodiment, a host cell transfected with a nucleic acid encoding an FKBP52 protein and a TRPC1 protein. The nucleic acid molecules encoding the an FKBP52 protein and a TRPC1 protein may be included in a nucleic acid encoding a fusion protein, wherein the nucleic acid encoding an FKBP52 protein or a TRPC1 protein is linked to a nucleic acid encoding another polypeptide. The fusion protein can be an FKBP52 polypeptide or a TRPC1 polypeptide linked to a readily detectible polypeptide. A “detectible polypeptide” is any polypeptide that can be readily identified using methods well known to one of skill in the art. In one embodiment, the detectible polypeptide can be an antigen which can be specifically bound by an antibody of interest (e.g., myc antigen and an anti-myc antibody). In another embodiment, the detectible polypeptide can catalyze an enzymatic reaction (e.g., lacZ). In yet another embodiment, the detectible polypeptide can be detected by its physical parameters (e.g., fluorescence when excited with light of a specific wavelength) or spatial parameters.

DNA sequences encoding an FKBP52 protein or a TRPC1 protein can be expressed in vitro by transfer of nucleic acid into a suitable host cell. “Host cells” are cells in which a vector can be propagated and its nucleic acid expressed. In a preferred embodiment, the host cell is eukaryotic. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, FKBP52 or TRPC1 encoding polynucleotide sequences may be inserted into an expression vector. The term “expression vector” refers to a plasmid, virus or other vehicle known in the art that has been manipulated by insertion or incorporation of the FKBP52 or TRPC1 nucleic acid sequences.

Polynucleotide sequences encoding FKBP52 and TRPC1 are available in public DNA databases. Full-length or functional fragments of these proteins may be used in the invention methods. Variant proteins, in particular those having conservative variations, having similar functional properties to the wild type proteins may also be used in the invention methods. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

Polynucleotide sequence which encode an FKBP52 or TRPC1 protein can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, as start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the invention (see e.g., Bitter et al., 1987, Methods in Enzymology 153:516-544). For example, when cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences encoding FKBP52 or TRPC1.

In the present invention, the polynucleotide encoding an FKBP52 or TRPC1 protein may be inserted into an expression vector which contains a promoter sequence which facilitates the efficient transcription of the inserted genetic sequence of the host. The expression vector typically contains an origin of replication, a promoter, as well as specific genes which allow phenotypic selection of the transformed cells. A specific, non-limiting example of a vectors suitable for use in the present invention include, but are not limited to the pMSXND expression vector for expression in mammalian cells (Lee and Nathans, 1988, J. Biol. Chem. 263:3521), amongst others. The DNA segment can be present in the vector operably linked to regulatory elements, for example, a promoter (e.g., T7, metallothionein I, neurofilament, or polyhedrin promoters).

Hosts can include yeast and mammalian organisms. Methods of expressing DNA sequences having eukaryotic or viral sequences in eukaryotes are well known in the art. In addition, prokaryotic cells, such as bacterial cells can be used for the production of FKBP52 or fragments thereof in the methods described herein. Biologically functional viral and plasmid DNA vectors capable of expression and replication in a host are known in the art. Such vectors are used to incorporate nucleic acid sequences of the use with the invention.

In yeast, a number of vectors containing constitutive or inducible promoters may be used. For a review see, Current Protocols in Molecular Biology, Vol. 2, 1988, Ed. Ausubel et al., Greene Publish. Assoc. & Wiley Interscience, Ch. 13; Grant et al., 1987, “Expression and Secretion Vectors for Yeast,” in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, “Heterologous Gene Expression in Yeast,” Methods in Enzymology, Eds. Berger & Kimmel, Acad. Press, N.Y., Vol. 152, pp. 673-684; and The Molecular Biology of the Yeast Saccharomyces, 1982, Eds. Strathem et al., Cold Spring Harbor Press, Vols. I and II. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. D. M. Glover, 1986, IRL Press, Wash., D.C.). Alternatively, vectors may be used which promote integration of foreign nucleic acid sequences into the yeast chromosome. Yeast may be used to search for molecules that disrupt binding between FKBP52 and TRPC1, for example, by co-expressing FKBP52 and TRPC1 or fragments thereof, that interact.

Mammalian expression systems which utilize recombinant viruses or viral elements to direct expression may be engineered. For example, when using adenovirus expression vectors, the FKBP52 or TRPC1 coding sequence may be ligated to an adenovirus transcription/translation control complex, e.g., the late promoter and tripartite leader sequence. Alternatively, the vaccinia virus 7.5K promoter may be used (e.g., see, Maced. et al., 1982, Proc. Natl. Acad. Sc. USA 79:7415-7419; Maced. et al., 1984, J. Biol. 49:857-864; Panniculi et al., 1982, Proc. Natl. Acad. Sc. USA 79:4927-4931). Of particular interest are vectors based on bovine papilloma virus which have the ability to replicate as extrachromosomal elements (Salver, et al., 1981, Mol. Cell. Biol. 1:486). Shortly after entry of this nucleic acid into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as, for example, the neo gene. Alternatively, the retroviral genome can be modified for use as a vector capable of introducing and directing the expression of the FKBP52 or TRPC1 gene in host cells (Cone & Mulligan, 1984, Proc. Natl. Acad. Sc. USA 81:6349-6353). High level expression may also be achieved using inducible promoters, including, but not limited to, the metallothionein I.A. promoter and heat shock promoters.

For long-term, high-yield production of recombinant proteins, stable expression is preferred. Rather than using expression vectors which contain viral origins of replication, host cells can be transformed with an FKBP52 or TRPC1, or fragment thereof, cDNA controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign nucleic acid, engineered cells may be allowed to grow for 1-2 days in an enriched media, and then are switched to a selective media. A number of selection systems may be used, including, but not limited to the herpes simplex virus thymidine kinase gene (Wigler, et al., 1977, Cell 11:223), hypoxanthine-guanine phosphoribosyltransferase gene (Szybalska & Szybalski, 1962, Proc. Natl. Acad. Sci. USA 48:2026), and the adenine phosphoribosyltransferase [Lowy, et al., 1980, Cell 22:817) genes can be employed in tk-, hgprt or aprt cells respectively. Additionally, antimetabolite resistance can be used as the basis of selection for which confers resistance to methotrexate (Wigler, et al., 1980, Natl. Acad. Sci. USA 77:3567; O'Hare, et al., 1981, Proc. Natl. Acad. Sci. USA 78: 1527); the gpt gene, which coders resistance to mycophenolic acid (Mulligan & Berg, 1981, Proc. Natl. Acad. Sci. USA 78:2072; the neo gene, which confers resistance to the aminoglycoside G-418 (Colberre-Garapin, et al., 1981, J. Mol. Biol. 150:1); and the hygro gene, which confers resistance to hygromycin (Santerre, et al., 1984, Gene 30:147) genes. Recently, additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.).

By “transformation” is meant a genetic change induce in a cell following incorporation of new DNA (i.e., DNA exogenous to the cell). Where the cell is a mammalian cell, the genetic change is generally achieved by introduction of the DNA into the genome of the cell (i.e., stable).

By “transformed cell” or “transfected cell” is meant a cell into which (or into an ancestor of which has been introduced, by means of recombinant DNA techniques, a nucleic acid molecule encoding an FKBP52, a TRPC1, or fragment thereof. Transformation of a host cell with recombinant DNA may be carried out by conventional techniques as are well known to those skilled in the art. Where the host is prokaryotic, such as E. coli, and is used for the production of an FKBP52 polypeptide, a TRPC1 polypeptide, or fragment thereof, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired. Vectors for the transformation of prokaryotic cells are well known to one of skill in the art (see Sambrook et al., supra).

When the host is a eukaryote, such methods of transfection of DNA as calcium phosphate co-precipitates, conventional mechanical procedures such as microinjection, electroporation insertion of a plasmid encased in liposomes, or virus vectors may be used. Eukaryotic cells can also be cotransformed with DNA sequences encoding an FKBP52 protein, a TRPC1 protein, or fragment thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

In another embodiment of the present invention, there are provided, methods of enhancing axonal outgrowth by inhibiting the activity of FKBP52 in a neuronal growth cone, thereby enhancing axonal outgrowth. In some embodiments, the activity of FKBP52 is inhibited by contacting the neuronal growth cone with an FKBP52 inhibitor. In certain embodiments, the FKBP52 activity inhibited is isomerase activity or binding of FKBP52 to TRPC1. In one aspect, FKBP52-TRPC1 signaling is inhibited.

In still another embodiment of the present invention, there are provided methods of enhancing axonal outgrowth in a subject in need thereof by administering an agent that inhibits the activity of FKBP52, thereby enhancing axonal outgrowth. In certain embodiments, the FKBP52 activity is isomerase activity or binding of FKBP52 to TRPC1. In one aspect, FKBP52-TRPC1 signaling is inhibited. In certain embodiments, the agent may be a peptide, a protein, an antibody, a polynucleotide, an antisense RNA molecule, an RNAi molecule, an siRNA molecule, a peptidomimetic, a chemical compound, a small organic molecule, or an agent from a library of test agents.

In certain embodiments, the subject has suffered an injury to the CNS. In other embodiments, the subject has a neurological disorder. In particular embodiments, the neurological disorder is selected from the group consisting of Alzheimer's, Parkinson's disease, senile dementia, memory disturbances/memory loss, Huntington's disease, Lou Gehrig's disease, multiple sclerosis, cerebral palsy, Creutzfeldt-Jakob disease, Niemann Pick disease, or Pick's disease.

In particular embodiments, the subject is administered a therapeutically effective amount of an agent that inhibits FKBP52 isomerase activity, binding to TRPC1, and/or FKBP52-TRPC1 signaling.

Delivery of the therapeutic polynucleotide that inhibits FKBP52-TRPC1 signaling can be achieved using a recombinant expression vector such as a chimeric virus or a colloidal dispersion system. In some embodiments for therapeutic delivery of antisense sequences, viral vectors or targeted liposomes may be used.

This invention involves administering to a subject a therapeutically effective dose of a pharmaceutical composition containing one or more agents that inhibit the binding of FKBP52 to TRPC1, FKBP52 isomerase activity, and/or FKBP52-TRPC1 signaling and a pharmaceutically acceptable carrier. “Administering” the pharmaceutical composition of the present invention may be accomplished by any means known to the skilled artisan.

The pharmaceutical compositions may be prepared and administered in dose units. Solid dose units are tablets, capsules and suppositories. For treatment of a subject or patient, depending on activity of the agent, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention are in general administered topically, intravenously, orally or parenterally or as implants, but other methods known to the skilled artisan may be used as well. Suitable solid or liquid pharmaceutical preparation forms are, for example, granules, powders, tablets, coated tablets, (micro) capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, 1990, Science, 249:1527-1533, which is incorporated herein by reference.

The pharmaceutical compositions according to the invention may be administered locally or systemically. By “therapeutically effective dose” is meant the quantity of an agent according to the invention necessary to prevent, to cure or at least partially arrest the symptoms of the disorder and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., 1990, Goodman And Gilman's: The Pharmacological Bases of Therapeutics, 8th ed., Pergamon Press; and Remington's Pharmaceutical Sciences, 1990, 17th ed., Mack Publishing Co., Easton, Pa., each of which is herein incorporated by reference.

The term “antibody” as used herein includes polyclonal and monoclonal antibodies, chimeric, single chain, and humanized antibodies, as well as intact molecules and fragments thereof, such as Fab, F(ab′)2, and Fv which are capable of binding to an epitopic determinant present in an FKBP52 protein. Such antibody fragments retain some ability to selectively bind with its antigen.

Methods of making antibodies and antibody fragments are known in the art. (See for example, Harlow and Lane, Antibodies: A Laboratory Manual, Cold Spring Harbor Laboratory, New York (1988), incorporated herein by reference). Monoclonal antibodies are made from antigen containing fragments of the protein by methods well known to those skilled in the art (Kohler & Milstein, Nature 256:495 (1975); Coligan et al., sections 2.5.1-2.6.7; and Harlow et al., Antibodies: A Laboratory Manual, page 726 (Cold Spring Harbor Pub. 1988), which are hereby incorporated by reference. Briefly, monoclonal antibodies can be obtained by injecting mice with a composition comprising an antigen/ligand, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG)” in Methods In Molecular Biology, Vol. 10, pages 79-104 (Humana Press 1992).

Antibodies that bind to FKBP52 and block FKBP52 binding to TRPC1 and/or FKBP52-TRPC1 signaling can be prepared using a portion of the FKBP52 protein where TRPC1 binds (e.g., the binding pocket of FKBP52) or a portion thereof as the immunizing antigen. For the preparation of polyclonal antibodies, the polypeptide or peptide used to immunize an animal is derived from translated cDNA or chemically synthesized and can be conjugated to a carrier protein, if desired. Commonly used carrier proteins which may be chemically coupled to the immunizing peptide include keyhole limpet hemocyanin (KLH), thyroglobulin, bovine serum albumin (BSA), tetanus toxoid, and the like.

Such polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See, for example, Coligan, et al., Unit 9, Current Protocols in Immunology, Wiley Interscience, 1994, incorporated herein by reference).

The following examples are intended to illustrate but not to limit the invention in any manner, shape, or form, either explicitly or implicitly. While they are typical of those that might be used, other procedures, methodologies, or techniques known to those skilled in the art may alternatively be used.

### Example

### Peptidyl-Prolyl Isomerase FKBP52 Controls Chemotropic Guidance of Neuronal Growth Cones Via Regulation of TRPC1 Channel Opening

The following example shows that the isomerase function of FKBP52 controls chemotropic guidance of neuronal growth cones in vitro and in vivo through regulation of TRPC1 channel opening.

Solutions, reagents and clones. Homer, TRPC1, FKBP12, and FKBP52 constructs were described previously. Point mutations were generated by site-directed mutagenesis (Stratagene). The antibodies used were monoclonal anti-myc and HRP-conjugated anti-myc and anti-HA (all from Santa Cruz Biotech), rabbit polyclonal anti-Homer 3, monoclonal anti-IP₃ receptor type 3 (for co-IP) (BD Biosciences), goat polyclonal anti-pan-IP₃ receptor (for Western blotting), rabbit polyclonal anti-FKBP59/FKBP52 (Affinity BioReagents), and monoclonal anti-mGluR1a (BD Biosciences). Plasmid transfection was done using calcium phosphate for 6 hours, washed once with 1×PBS, and replaced with regular HEK293 media. Cells were incubated at 37° C. for 36-48 hours. The amount of cDNA transfected in a 100-mm dish was 10 μg or less, as indicated in the figure legends. Current was measured with electrophysiology, or cells were harvested and extracted for co-IP analysis the following day.

GST pull-down, Co-IP and immunocytochemistry. Protein binding assays were done as previously described (Yuan et al., Cell 114:777-89, 2003). Transfected cells were harvested and lysed using 500 μl of binding buffer (1×PBS buffer containing 1 mM NaVO3, 10 mM NaPyrophosphate, 50 mM NaF [pH 7.4], and 1% Triton X-100). The cell extracts were sonicated, and insoluble material was spun down at 30,000 g for 20 min. For GST pull-down, GST-H3 EVH was expressed in BL21 bacterial cells by growing a 400 ml culture at 37 C until A595 was 0.4-0.6. Cells were induced with 125 mM IPTG for an additional hour, harvested, lysed in 10 μl of 1% Triton X-100 in 1×PBS+200 mg/ml PMSF, sonicated 3×10 strokes, and spun down at 12,000 g for 5 min. The supernatant was mixed 1:1 with a slurry of GST-agarose beads (Sigma) for 30 min at 4 C. Beads were washed 2 times with 1% Triton X-100 in 1×PBS and 2 times with 1×PBS. 50 μl of 1:1 slurry of GST-H3 EVH beads were used per 100 μl cell extract and rocked for 2 hr at 4° C. For co-IP experiments, 1 μg of purified antibody or 2 μl of crude antiserum was added to 100 μl of cell extract and incubated for 1 hr at 4° C. Then, 1:1 slurry of protein A or G SEPHAROSE 4B beads were added to the antibody-extract mix and incubated for an additional hour at 4° C. Beads were washed 3×10 min with binding buffer, and proteins were released from the beads with SDS-loading buffer and subjected to western blot analysis.

For the FK506 time response assay, FK506 was added directly to the media of transfected cells to a final concentration of 150 nM at various time points: 15, 30 min; 1, 3, 6, 12, 24 hrs before cells were harvested. Extracts were made and used for GST-Homer pull-down assay. For 3-(3-pyridyl)-1-propyl (2S)-1-(3,3-dimethyl-1,2-dioxopentyl)-2-pyrrolidinecarboxylate (GPI-1046) dosage response assay, GPI-1046 was administered for 30 min directly to the media 24 hrs after transfection, and lysates were assayed for GST-Homer pull-down.

Biotinylation assay. Transfected cells were washed once with 1×PBS on ice. EZ-Link Sulfo-NHS-SS-Biotin (0.5 mg/ml; Pierce) was added to the cells for 30 min on ice. Afterwards, the biotin was quenched with 50 mM glycine on ice for 10-15 min. The cells were then processed as described above to make cell extract. 50 μl of 1:1 slurry of immobilized avidin beads (Pierce) were added to 100 μl of cell extract and incubated for 2 hrs at 4° C. Beads were washed 3×10 min with binding buffer and proteins were released from the beads with SDS-loading buffer and subjected to western blot analysis.

Electrophysiology analysis of TRPC1 current in HEK293 cells. TRPC1 current in HEK293 cells was measured with/without over-expression of WT or mutant FKBP12/FKBP52 in a whole-cell current recording configuration, as described previously (Huang et al., Nat Cell Biol 8:1003-10, 2006; and Yaun et al., Nat Cell Biol 9:636-45, 2007). Briefly, the pipette solution contained (in mM) 140 CsCl, 2 MgCl₂, 1 ATP, 5 EGTA, 1.5 CaCl₂ (free Ca²⁺ at 70 nM) and 10 HEPES at pH 7.2 with CsOH, to eliminate K⁺ current and prevent inhibition of the channels by high cytoplasmic Ca²⁺. The bath solution contained (in mM) 140 NaCl or 140 NMDG-Cl, 5 KCl, 0.5 EGTA and 10 HEPES at pH 7.4 with NaOH or NMDG-OH. The current was measured by ramp from −100 mV to +100 mV for 400 ms every 4 s at holding potential 0 mV. The current recorded at −100 mV was used to calculate current density as pA/pF. Multiple independent experiments were used to obtain the mean±s.e.m. Statistical significance was assessed using Student's t-test.

Whole-mount in situ hybridization. Whole-mount in situ hybridization was carried out using the digoxigenin (DIG)-UTP-labelled antisense and sense RNA probe as previously described (Harland, Methods Cell Biol 36:685-95, 1991). The C-terminal open reading frame regions of XFKBP52 (BamHI-stop codon fragment) and XTRPC1 (EcoRI-stop codon fragment) were used for the specific anti-sense and sense RNA probes. The labelled probes were detected with alkaline phosphatase-conjugated anti-DIG antibody (Fab fragments) and visualized with the BM purple AP substrate (Roche Applied Science).

Xenopus embryo injection and cell culture. Blastomere injections of morpholino oligos or mRNAs encoding TRPC1, FKBP52 or their mutant forms into early stages of Xenopus embryos and culturing of spinal neurons from these injected embryos were performed as previously described (Shim et al., Nat Neurosci 8:730-5, 2005). Specifically, fertilized embryos were injected at the one- or two-cell stage, with a mixture of the morpholino (10 ng/embryo) or mRNA (2-3 ng/embryo), and a lineage tracer. The following DNA constructs were subcloned into the pCS2 vector and used for in vitro transcription with the mMESSAGE mMACHINE SP6 kit (Ambion): Myc-FKBP52-WT, Myc-FKBP52-FD67DV, HA-TRPC1-WT, HA-TRPC1-P645L, HA-TRPC1-P645A, GFP-XTRP C1. A morpholino oligo directed against Xenopus DCC (XDCC-MO) was designed with the following sequence: 5′ CCAAGACAATTCTCCATATTTCAGC 3′. Injected embryos at stage 22 were used for cultures of spinal neurons as previous described (Shim et al., Nat Neurosci 8:730-5, 2005).

Primary hippocampal neuronal cultures and transfection. Hippocampal neurons were isolated from postnatal rats (P3-P5) and were cultured on poly-L-lysine coated coverslips as previously described (Song et al., Nature 417:39-44, 2002). These neurons were transfected with the AMAXA transfection system following protocols from the manufacturer. Briefly, hippocampal neurons were isolated and 100 μl of nucleofector solution was added to resuspend the cell pellet. Different expression constructs (1-5 μg), GFP, FKBP52-FD67DV, TRPC1-P645A or a combination of TRPC1-P645A and FKBP52-WT, were added to the cell suspension and then transferred to cuvettes for electroporation. The cells were cultured in DMEM with 10% fetal bovine serum for 24 hrs before changing to serum-free neurobasal medium (Song et al., Nature 417:39-44, 2002).

Ca²⁺ imaging of cultured spinal neurons. Ca²⁺ imaging of growth cones of Xenopus spinal neurons was carried out as previously described (Ming et al., Nature 417:411-8, 2002; and Ming et al., Neuron 19:1225-35, 1997). Specifically, isolated Xenopus spinal neurons were loaded with Fluo-4 AM (2 μM, Molecular Probes) in the culture media without serum for 30 min at room temperature, and rinsed with the media for growth cone turning assay. Ca²⁺ imaging was performed using a Zeiss 510 META system equipped with a 20× objective (NA 0.8). Excitation was at 488 nm by argon laser and the emitted fluorescence was collected at 500-560 mm. Fluorescence and bright-field images were simultaneously acquired at every 30 seconds with a frame scan. The fluorescence intensity of each time point was measured over a fixed rectangular region of interest that covers the entire growth cone and normalized to the average fluorescence intensity that was measured during a 5 min baseline period (prior to netrin-1 application).

Growth cone turning assay. Microscopic gradients of netrin-1 (5 μg/ml in the pipette), MAG (150 μg/ml in the pipette) and GPI-1046 (100 μM in the pipette) were produced as previously described (Lohof et al., J Neurosci 12:1253-61, 1992; and Zheng et al., Nature 368:140-4, 1994). Previous characterization showed that at 100 μm away from the pipette tip the concentration of factors is about 1000 fold lower than that in the pipetter and there is a 5-10% concentration gradient across the width of the growth cone. Xenopus spinal neurons were used 14 to 20 hrs after plating and neurons with the lineage-tracer were identified under fluorescent microscope and used for turning assay at room temperature as previously described (Ming et al., Nature 417:411-8, 2002; Ming et al., Neuron 19:1225-35, 1997; Lohof et al., J Neurosci 12:1253-61, 1992; and Zheng et al., Nature 368:140-4, 1994). GFP⁺ hippocampal neurons were used 48-72 hours after plating and individual axon was identified for turning assay at the room temperature. The turning angle was defined by the angle between the original direction of neurite extension and a straight line connecting the positions of the center of the growth cone at the onset and the end of the 30 min period. The rates of neurite extension were calculated based on the net neurite extension during the turning assay. Only those growth cones of isolated neurons with a net neurite extension >5 μm over the 30-min period were included for analysis. Statistical significance was assessed using the Kolmogorov-Smirnov test.

Whole-mount immunocytochemistry and confocal imaging. Embryos at stage 23-28 were fixed and processed for immunocytochemistry as previously described (Shim et al., Nat Neurosci 8:730-5, 2005; and Gomez et al., Nature 397:350-5, 1999). Monoclonal antibody 3A10, specific for commissural interneurons, was obtained from the Developmental Studies Hybridoma Bank at the University of Iowa and used at a dilution of 1:100. Secondary antibodies were used at a dilution of 1:250. Confocal images of sagittal views of embryos were taken with a Zeiss LSM 510 META system and Z-series reconstructions were processed with the Zeiss LSM image acquisition program. A minimum of 10 embryos was examined for each condition. Statistical significance was assessed using the Bootstrap test and accepted if p <0.01.

Biochemical characterization of interaction between FKBP52 and TRPC1. To explore the potential molecular mechanism of FKBPs in regulating TRPC1 channel activity, biochemical analysis of protein-protein interaction was first carried out. In particular, TRPC1-WT (wild-type) co-immunoprecipitated (co-IP) with FKBP52 when co-transfected into HEK293 cells (FIG. 1b). Furthermore, point mutations within the TRP box 2 of TRPC1 (including TRPC1-P645L, P646L and F648R) disrupted the ability of TRPC1 to co-IP with FKBP52 (FIG. 1b). Homer was previously shown to bind to TRPC1 and inhibits the channel opening (Yuan et al., Cell 114:777-89, 2003). Interestingly, these mutations of TRPC1 also disrupted binding to Homer (FIG. 1b), suggesting that FKBP52 and Homer bind to the same region in TRPC1. Additional screens identified mutants of TRPC1 (including TrpC1-L644S, L644A and P645A) that only bound FKBP52 but did not bind Homer (FIG. 1b). TRPC1 possesses a second Homer binding site in the N-terminus with a minimal consensus for FKBP binding (FIG. 1a). However, point mutations in the N-terminus of full-length TRPC1 that disrupt its co-IP with Homer was not altered (Supplementary FIG. 1). Since FKBP52 binding to the C-terminus could obscure effects of mutations in the N-terminus, binding studies were repeated using an N-terminal fragment of TRPC1 (TRPC1-NT). GST-FKBP52 showed direct association with WT-TRPC1-NT and TRPC1-NT-P23A, but not TRPC1-NT-L19A or TRPC1-NT-P20A (FIG. 1c). FKBP12, a lower molecular weight homologue of FKBP52, did not bind TRPC1 in co-IP or GST pull-down assays. These data indicated that FKBP52 and Homer bind the same regions of TRPC1 but possess distinct sequence requirements.

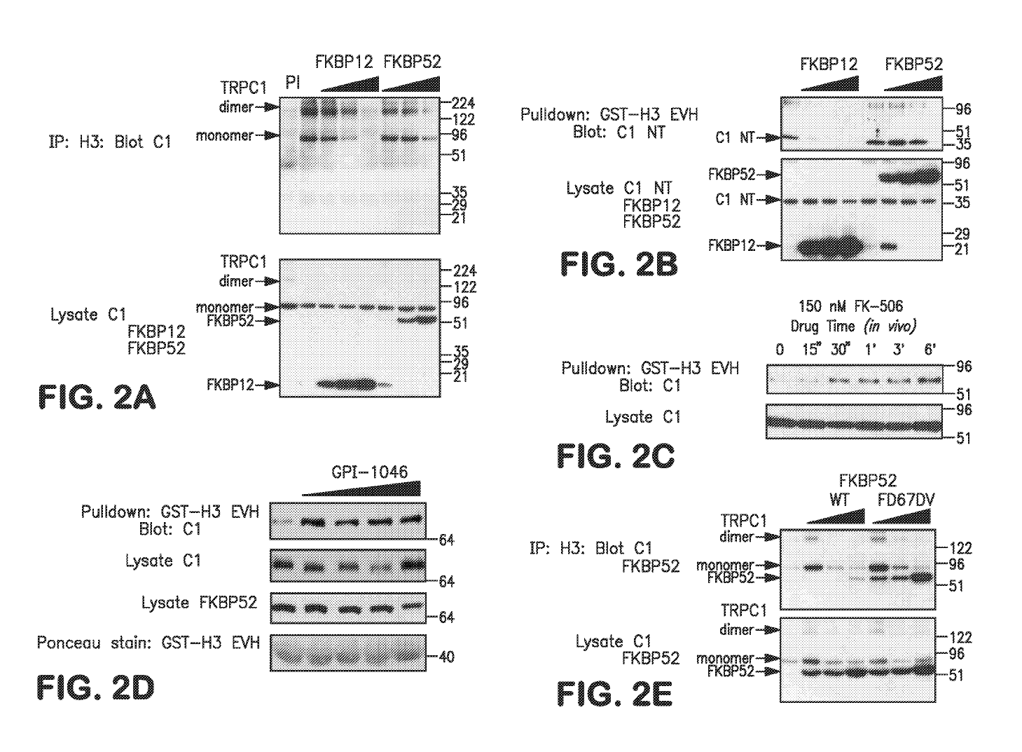

The overlapping binding sites in TRPC1 suggested that Homer and FKBP52 might compete with each other. In support of this notion, increasing FKBP52 expression reduced the amount of TRPC1 that co-IPed with native Homer 3 (FIG. 2a). Identical inhibition of Homer binding to TRPC1 was obtained with FKBP12 (FIG. 2a). Since FKBP12 does not show binding to TRPC1 in co-IP or pull-down assays, these results suggested that FKBP12 interacted with TRPC1, but dissociated rapidly in binding assays. FKBP52 and FKBP12 exhibited similar inhibition on GST-Homer binding to TRPC1 or TRPC1-NT in vitro (FIG. 2b and Supplementary FIG. 2a). The competing effect of FKBPs and Homer was specific for TRPC1 as they did not alter TRPC1 binding to the metabotropic glutamate receptor mGluR1a (Supplementary FIG. 2b). As a further test of competitive binding, HEK293 cells were treated with FK506 to dissociate native FKBP52. Interestingly, FK506 treatment led to rapid (within 30 min) and sustained (through 6 hrs) enhancement of TRPC1 binding to GST-Homer (FIG. 2c). To confirm that the action of FK-506 was due to inhibition of FKBPs, the compound GPI-1046, which binds FKBPs and inhibits their isomerase activity, but unlike FK-506, does not inhibit calcineurin (Steiner et al., PNAS 94:2019-24, 1997) was tested. Indeed, treatment of HEK293 cells with GPI-1046 also increased TRPC1 binding to GST-Homer (FIG. 2d). To evaluate the role of isomerase activity of FKBPs in binding assays, FKBP point mutants FKBP52-FD67DV and FKBP12-D37L that lack isomerase activity (Barent et al., Mol Endocrinol 12:342-54, 1998; and Schiene-Fischer et al., FEBS Lett 495:1-6, 2001) were generated. Both mutants inhibited Homer co-IP of TRPC1 with an efficacy similar to FKBP-WT (FIG. 2e and Supplementary FIG. 2c). Thus, isomerase activity of FKBPs was not required for competitive binding. While the present result was consistent with simple bimolecular competition, it was noted that FKBP52-FD67DV co-IPed with Homer 3 (FIG. 2e), suggesting that binding dynamics may also involve allosteric effects.

While not wishing to be bound by any particular theory, these biochemical findings when taken together, support a model that FKBPs compete with Homer, a regulator of TRPC1 channel opening, for binding to TRPC1 in an isomerase activity-independent fashion.

Isomerase activity-dependent regulation of ligand-induced TRPC1 channel opening by FKBP52. The effect of FKBP52 on TRPC1 function was next examined by monitoring membrane surface expression and channel activity of TRPC1. Expression of both WT and isomerase inactive mutants of FKBPs modestly increased TRPC1 on the membrane surface (˜2 fold), without changing expression in the lysates (Supplementary FIG. 3). The physical association of TRPC1 with IP₃R, which has been implicated in channel opening, was not altered by FKBPs (Supplementary FIG. 3). Similarly, the association of Homer and IP₃R, which may be important for internal Ca²⁺ release, was also unaffected by FKBPs (Supplementary FIG. 3). Recordings of TRPC1 currents from the same preparation, however, revealed striking differences in the spontaneous and agonist-induced currents. Under the basal condition, TRPC1 produced a small spontaneous inward current that increased ˜2 fold upon stimulation of native purinergic receptors with UTP (FIGS. 3a & 3f). Co-expression of FKBP12 resulted in ˜6 fold increase in the total current (FIGS. 3b & 3f). Most of the current was spontaneously active since it was not further increased by stimulation with UTP (FIGS. 3b & 3f). Expression of FKBP52 also resulted in a marked increase of the total current (FIGS. 3d & 3f). In contrast to FKBP12, the major effect of FKBP52 was due to an increase of the agonist-stimulated current with little effect on the spontaneous current (FIGS. 3d & 3f). Importantly, the effects of both FKBP12 and FKBP52 on TRPC1 were dependent on the isomerase activity since FKBP12-D37L and FKBP52-FD67DV blocked both the spontaneous and UTP induced currents (FIGS. 3c, 3e & 3f). Further analysis showed that TRPC1 mutants P645A and P23A, which bind FKBP52 but not homer (FIG. 1a & supplementary FIG. 1), were constitutively active without ligand (Supplementary FIG. 4). Interestingly, co-expression of FKBP52 with these mutants restored the ligand-induced current and reduced the spontaneous current without increasing the total current (Supplementary FIG. 4). Taken together, these electrophysiological results indicated that FKBP52 amplifies the ligand-induced activation of TRPC1 channel in an isomerase activity-dependent fashion.

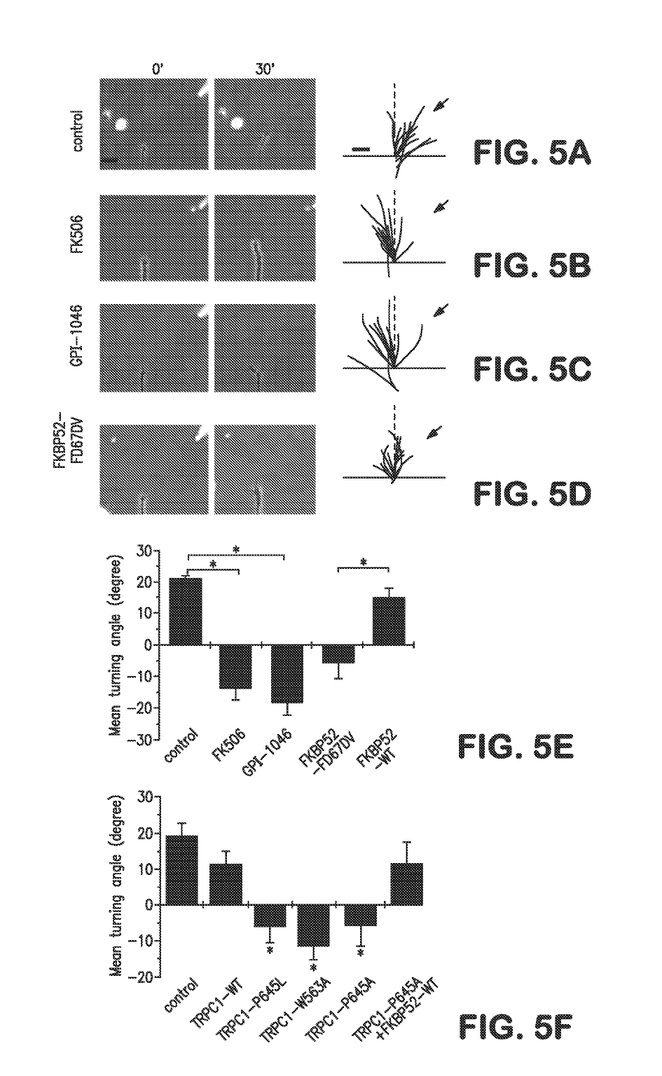

Requirement of isomerase activity of FKBP52 in netrin-1-induced Ca²⁺ rise in neuronal growth cones. FKBP52 is conserved between species and highly expressed in the developing nervous system (Supplementary FIG. 5). Given the specific role of FKBP52 in regulating ligand-induced TRPC1 opening, analysis was focused on its function in neurons. XTRPC1 has been identified as a critical mediator of netrin-1-induced Ca²⁺ influx and growth cone guidance (Wang et al., Nature 434:898-904, 2005; and Shim et al., Nat Neurosci 8:730-5, 2005). As reported, netrin-1 induced significant Ca²⁺ rise within growth cones of cultured Xenopus spinal neurons (FIGS. 4a, 4d & 4e) (Ming et al., Nature 417:411-8, 2002). This Ca²⁺ rise required netrin-1 receptor DCC, since a morpholino specifically against Xenopus DCC (XDCC-MO) abolished netrin-1-induced Ca²⁺ rise (FIGS. 4d & 4e). Interestingly, isomerase inhibition by GPI-1046 (100 nM, FIG. 4b) or over-expression of FKBP52-FD67DV (FIG. 4c) also abolished netrin-1-induced Ca²⁺ rise in growth cones (FIGS. 4d & 4e). Furthermore, over-expression of a mutant form of TRPC1 (P645L) that does not bind to FKBP52 (FIG. 1b) while maintains its basal channel activity, abolished netrin-1-induced Ca²⁺ rise (FIG. 4e). These results suggested that FKBP52 regulates netrin-1-induced Ca²⁺ elevation in neuronal growth cones via proline isomerase activity-dependent regulation of the TRPC1 function.

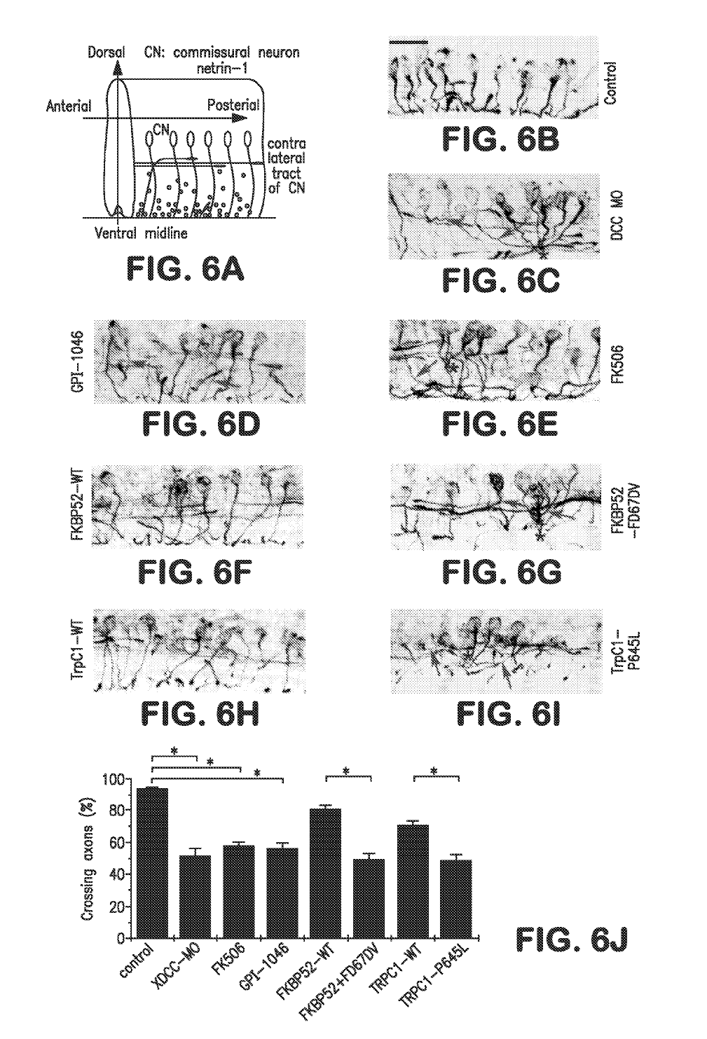

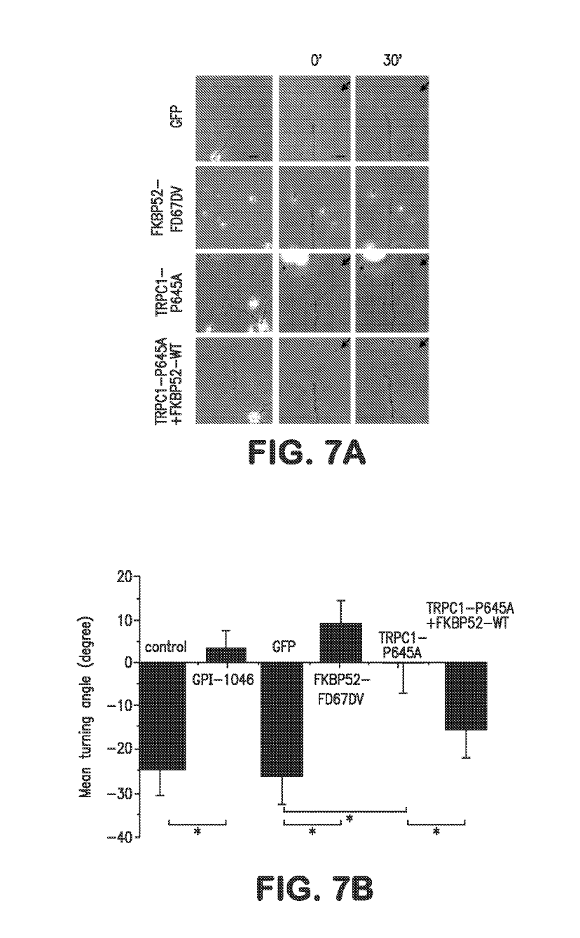

Requirement of isomerase activity of FKBP52 in netrin-1-induced growth cone turning. The role of proline isomerase in axon guidance was directly assessed using a well-established in vitro growth cone turning assay (Ming et al., Nature 417:411-8, 2002; Ming et al., Neuron 19:1225-35, 1997; Lohof et al., J Neurosci 12:1253-61, 1992; Song et al., Science 281:1515-8, 1998; and Zheng et al., Nature 368:140-4, 1994). In a microscopic gradient of netrin-1, Xenopus spinal neurons exhibited chemoattractive turning responses (FIG. 5a). When these neurons were exposed to a netrin-1 gradient in the presence of FK506, attractive growth cone turning responses were completely abolished (FIG. 5b, 5e & Supplementary FIG. 6a). Application of GPI-1046 also blocked netrin-1-induced attraction (FIG. 5c, 5e & Supplementary FIG. 6b). Growth cones were also subjected to a gradient of GPI-1046 (100 μM in the pipette) with concurrent and uniform netrin-1 activation (10 ng/ml in the bath) to generate a reverse gradient of isomerase activity. Interestingly, neuronal growth cones exhibited significant repulsive turning responses under this condition (Supplementary FIG. 7). In contrast, neurons in the same GPI-1046 gradient without a uniform netrin-1 stimulation exhibited no bias in the direction of growth cone extension (Supplementary FIG. 7). Taken together, these results suggested that proline isomerase activity was part of the instructive mechanism underlying growth cone steering induced by netrin-1.

The role of isomerases in growth cone guidance was further characterized with molecular genetic approaches. Over-expression of FKBP52-FD67DV, but not FKBP52-WT, in Xenopus spinal neurons abolished attractive turning responses to netrin-1 (FIGS. 5d & 5e), resembling those with lower doses of FK506 and GPI-1046 (Supplementary FIG. 6). In addition, expression of TRPC1-P645L, but not TRPC1-WT, also abolished netrin-1-induced attraction (FIG. 5f), similar to over-expression of FKBP52-FD67DV or a channel pore mutant TRPC1-W563A (FIG. 5f). Interestingly, expression of TRPC1-P645A, which showed constitutive channel activity in HEK293 cells (Supplementary FIG. 4), also abolished netrin-1-induced attraction (FIG. 5f). Importantly, over-expression of FKBP52-WT rescued netrin-1-induced attraction (FIG. 5f). Taken together, these results suggested that proline isomerase activity-dependent regulation of TRPC by FKBP52 is required for growth cone steering induced by netrin-1.

Requirement of FKBP52 and its regulation of TRPC1 in commissural axon guidance in vivo. The question of whether proline isomerase activity of FKBP52 was also required for axon guidance in vivo was examined. Netrin-1/DCC signalling is known to guide commissural axons to the CNS midline in the developing spinal cord (FIG. 6a). A specific morpholino against XDCC was injected into Xenopus embryos at the one-cell stage and examined stage 25 embryos, a time when commissural interneuron axons have already reached the CNS midline. Commissural interneuron axons in developing Xenopus embryos were specifically identified with the 3A10 monoclonal antibody (FIG. 6b). Many of the 3A10⁺ axons in embryos with XDCC-MO failed to reach the midline; instead, they joined ipsilateral tracts or turned longitudinally between the ipsilateral tract and the midline (FIG. 6c). Interestingly, some of the commissural axons also lost contact avoidance and exhibited fasciculation prematurely before crossing the midline (FIG. 6c). Thus, netrin-1/DCC-dependent midline guidance is also functionally conserved in developing Xenopus embryonic spinal cord.

Next, GPI-1046 (0.5 μM) or FK506 (0.5 μM) were applied to developing Xenopus embryos between stage 22-25, a time window when commissural interneuron axons are attracted to the midline. Such treatments led to significant guidance defects as those with XDCC-MO (FIGS. 6d, 6e & 6j). Over-expression of FKBP52-FD67DV, but not WT-FKBP52, led to similar midline targeting errors and premature fasciculation (FIGS. 6f, 6g & 6j). Strikingly, some of the commissural neurons expressing FKBP52-FD67DV appeared to completely ignore guidance cues and sent long projections to the notochord. Finally, expression of TRPC1-P645L, but not TRPC1-WT, led to significant midline guidance defects (FIGS. 6h, 6i & 6j), which were very similar to those resulted from XDCC knockdown, inhibition of FKBP52 isomerase activity, or expression of a TRPC1 channel pore mutant (F562A) as previously shown (Shim et al., Nat Neurosci 8:730-5, 2005). Taken together, these results demonstrated that isomerase-dependent regulation of TRPC1 by FKBP52 is required for commissural interneuron axon guidance in the developing Xenopus spinal cord in vivo.

Requirement of isomerase activity of FKBP52 in growth cone responses to MAG. Ca²⁺ signalling has been implicated in the signalling of myelin and glia scar associated inhibitors that prevent axonal regeneration in the adult central nervous system (Song, H. et al., Science 281, 1515-8, 1998; Henley et al., Neuron 44, 909-16, 2004; Sivasankaran et al., Nat Neurosci 7, 261-8, 2004; and Koprivica et al., Science 310, 106-10, 2005). Myelin-associated glycoprotein (MAG) is a major component of myelin and it was showed that MAG-induced repulsion of growth cones of Xenopus spinal neurons requires function of XTRPC1. Similar to Xenopus spinal neurons, axonal growth cones of rat hippocampal neurons exhibited significant repulsive turning responses in a MAG gradient (FIG. 7a). Interestingly, application of GPI-1046 or over-expression of FKBP52-FD67DV abolished MAG-induced repulsion in these neurons (FIGS. 7a & 7b). Furthermore, over-expression of TRPC1-P645A, which shows constitutive channel activity in HEK293 cells (Supplementary FIG. 4), also blocked MAG-induced growth cone repulsion (FIGS. 7a & 7b). Importantly, co-expression of FKBP52-WT rescued MAG-induced growth cone repulsion in neurons (FIG. 7). Taken together, these results suggested that proline isomerasation of TRPC1 by FKBP52 was also essential for transducing MAG signalling.

Although the invention has been described with reference to the above examples, it will be understood that modifications and variations are encompassed within the spirit and scope of the invention. Accordingly, the invention is limited only by the following claims.

What is claimed is:

1. A method of identifying an agent that inhibits FKBP52 binding to TRPC1 comprising:
comparing binding of FKBP52 to TRPC1 in the presence and absence of a test agent, wherein a decrease in binding in the presence of test compound is indicative of an agent that is an inhibitor of FKBP52 binding.

2. The method of claim 1, wherein the FKBP52 and TRPC1 are expressed in the same cell.

3. The method of claim 2, wherein the cell is transfected with a nucleic acid molecule encoding FKBP52, a nucleic acid molecule encoding TRPC1, or both.

4. The method of claim 3, wherein binding is determined by co-immunoprecipitation of FKBP52 and TRPC1.

5. The method of claim 1, wherein the test agent is selected from the group consisting of a peptide, a protein, an antibody, a polynucleotide, an antisense RNA molecule, an RNAi molecule, an siRNA molecule, a peptidomimetic, a chemical compound, and a small organic molecule.

6. The method of claim 1, wherein the test agent is one of a library of test agents.

7. A method of detecting or identifying an inhibitor of an FKBP52-TRPC1 signaling pathway comprising:
contacting a cell expressing TRPC1 and FKBP52 with a test agent and detecting a change in agonist-induced calcium flux of TRPC1, wherein a decrease in calcium flux in the presence of the test agent as compared to in the absence of test agent, is indicative that the agent is an inhibitor of FKBP52-TRPC signaling.

8. The method of claim 7, wherein the TRPC1 is activated by activation of a G-protein coupled receptor (GPCR).

9. The method of claim 8, wherein the GPCR is activated by an agonist.

10. The method of claim 8, wherein the GPCR is a purinergic receptor.

11. The method of claim 10, wherein the agonist is UTP.

12. The method of claim 7, wherein the cell is an HEK293 cell.

13. The method of claim 7, wherein the change in agonist-induced calcium flux is detected as a change in current.

14. The method of claim 7, further comprising determining the effect of the test agent the binding of FKBP52 to TRPC1.

15. An inhibitor of FKBP52, identified by the method of claim 1.

16. A method of enhancing axonal outgrowth comprising:
inhibiting the activity of FKBP52 in a neuronal growth cone, thereby enhancing axonal outgrowth.

17. A method of enhancing axonal outgrowth in a subject in need thereof comprising:
administering an agent that inhibits the activity of FKBP52, thereby enhancing axonal outgrowth.

18. The method of claim 17, wherein the activity inhibited is isomerase activity.

19. The method of claim 17, wherein the activity inhibited is binding of FKBP52 to TRPC1.

20. The method of claim 17, wherein FKBP52-TRPC1 signaling is inhibited.

21. The method of claim 17, wherein the agent is selected from the group consisting of a peptide, a protein, an antibody, a polynucleotide, an antisense RNA molecule, an RNAi molecule, an siRNA molecule, a peptidomimetic, a chemical compound, and a small organic molecule.

22. The method of claim 17, wherein the subject has suffered an injury to the CNS.

23. The method of claim 17, wherein the subject has suffered an iatrogenic injury to a nerve during surgery.

24. The method of claim 23, wherein the surgery is prostate surgery.

25. The method of claim 17, wherein the subject has a neurological disorder.

26. The method of claim 25, wherein the neurological disorder is a demyelinating disease.

27. The method of claim 26, wherein the demyelinating disease is multiple sclerosis.

28. The method of claim 25, wherein the neurological disorder is selected from the group consisting of Alzheimer's, Parkinson's disease, senile dementia, memory disturbances/memory loss, Huntington's disease, Lou Gehrig's disease, multiple sclerosis, cerebral palsy, Creutzfeldt-Jakob disease, Niemann Pick disease, and Pick's disease.
